(19) United States
(12) Patent Application Publication
(10) Pub. No.: US 2012/0137390 A1
(43) Pub. Date: May 31, 2012

(54) MATERIALS AND METHODS FOR REGENERATION AND TRANSFORMATION OF TREES

(75) Inventors: Shujun Chang, Wilmington, DE (US); Heather Gladfelter, Gainesville, GA (US); Eric Gulledge, Charleston, SC (US)

(73) Assignee: ArborGen Inc.

(21) Appl. No.: 13/258,826

(22) PCT Filed: May 6, 2010
(86) PCT No.: PCT/US10/33821

§ 371 Date: Nov. 14, 2011

Related U.S. Application Data
(60) Provisional application No. 61/176,314, filed on May 7, 2009.

(51) Int. Cl. C12N 15/82; C12N 5/04

(52) U.S. Cl. 800/294; 435/430; 435/419; 435/430.1

(57) ABSTRACT

The present disclosure relates to a method for regenerating shoots from plant explants using a plant culture medium including meta-topolin. The invention also provides media and methods for regenerating plants, particularly forest trees. In particular, methods for regenerating stably transformed Eucalyptus and pine trees are provided.

### CROSS-REFERENCE TO RELATED APPLICATIONS

This application claims priority to U.S. Provisional Application No. 61/176,314, filed May 7, 2009, the entire contents of which are hereby incorporated by reference in its entirety.

### TECHNICAL FIELD

The present technology relates generally to the field of plant tissue culture. In particular, the technology provides methods for increasing shoot regeneration efficiency from recalcitrant clones or genotypes of elite tree species.

### BACKGROUND

The following description is provided to assist the understanding of the reader. None of the information provided or references cited is admitted to be prior art to the present invention.

Plant genetic engineering has provided great potential for the improvement of commercially important plant species. In recent years, the genetic engineering of trees has gained momentum and finds particular application in the pulping and timber industries. Several established tree transformation systems exist for such species as sweetgum, European larch, yellow poplar and many Populus species. Various traits, such as insect resistance and herbicide tolerance, have been engineered into these tree species. Thus, the potential for genetically engineering tree species is great for commercially important tree species, including Eucalyptus, pine and sweetgum.

Eucalyptus plants are polygenus plants comprising more than five hundred species. Eucalyptus has a high growth rate, adapts to wide range of environments, and displays little susceptibility to insect damage. In addition to its exceptional growth properties, Eucalyptus trees provide the largest source of fibers for the paper industry. Fibers from hardwood species, such as Eucalyptus, are generally much shorter than fibers from softwoods, such as pine. The shorter fibers produced from Eucalyptus results in the production of pulp and paper with desirable surface characteristics, including smoothness and brightness, but low tear or tensile strength. As a timber, Eucalyptus provides tall, straight timber with a medium to high density. Eucalyptus timber is general-purpose; finds use in the plywood and particleboard industry, furniture industry; and provides a source of firewood and construction materials.

Most reports demonstrating transformation of Eucalyptus use Eucalyptus seedlings rather than elite genotypes or clones obtained through breeding programs. For example, WO 99/48355 describes a method for transforming young leaf explants from seedlings of E. grandis and E. camaldulensis. Even though the transformation was successful, there are two main problems with the described method. First, the regeneration protocol works for seedling explants, but it does not work with explants from elite genotypes. Second, even with seedling explants and the claimed improvements, the transformation efficiency is limited to 2.2% or lower for cotyledon explants of the two species and the hypocotyl explants of E. camaldulensis. An improved transformation and regeneration protocol was reported for E. camaldulensis seedlings by Ho et al., Plant Cell Reports 17:675-680 (1998); but, the protocol was not repeatable even with E. camaldulensis seedlings. Thousands of explants were cultured, and transgenic callus lines were produced, but the number of shoots recovered was minimal.

Although micropropagation of Eucalyptus clonal materials has been performed routinely, de novo shoot regeneration has been limited to seedlings instead of the selected or “elite” clones of commercially important Eucalyptus species. Elite genotypes, which arise through successive rounds of breeding and selection, are valued for their combination of economically desirable traits. Unlike seedling transformation, which requires a large number of genotypes to ensure co-segregation of growth traits with the desired trait conferred by transgene expression, the transformation of elite clones would provide an efficient and advantageous system for genetically engineering tree species. Elite genotypes can be selected based on many years of clonal field tests with a large number of starting genotypes. Like many other fast growing hardwood tree species, it takes years of field evaluation before relatively accurate predictions of a trait can be made for Eucalyptus. Therefore, if seedlings are used for genetic engineering, an even larger number of genotypes is needed for successful selection of a growth trait together with a desired trait conferred by transgene expression.

### SUMMARY

For elite genotypes or clones of trees, such as Eucalyptus, it is often difficult to regenerate shoots from explant tissues. This lack of regeneration or low regeneration efficiency has prohibited genetic transformation of these elite genotypes or clones because regeneration is required for selection and development of transgenic cells.

Conventionally, shoot regeneration for tree species has been achieved by using growth regulators such as BAP, zeatin, kinetin, 2ip, and TDZ. However, for certain elite lines, none of the mentioned growth regulators or combinations were successful at inducing shoot regeneration either alone or in combination. The methods described herein demonstrate ways to overcome the regeneration problem for elite trees, such as Eucalyptus, that are selected from breeding programs.

The present inventors discovered the media components that are important for regenerating shoots from elite genotypes or clones. Thus, the present invention generally relates to the use of N⁶-(meta-hydroxybenzyl)adenine (meta-topolin) and combinations of meta-topolin with other plant growth regulators to promote the regeneration of shoots of elite tree species that are otherwise difficult to regenerate. While the use of meta-topolin alone or in combination with other growth regulators are effective to induce regeneration from certain elite genotypes, these compounds are anticipated to improve the regeneration efficiency of other genotypes as well.

In one aspect, the disclosure describes a method for regenerating a plant from plant tissues comprising: (a) culturing plant tissue in a medium that comprises (i) meta-topolin and (ii) one or more additional cytokinins, wherein at least one cytokinin is selected from the group consisting of: thidiazuron (TDZ) and N-(2-Chloro-4-pyridyl)-N′-phenylurea (4-CPPU); and (b) incubating the plant tissue until one or more shoots or shoot primordia are formed.

In one embodiment, the plant tissue is an explant selected from the group consisting of: a leaf explant, a petiole explant, an internode explant, a floral tissue explant, and an embryogenic tissue explant. For example, the plant tissue may be an explant that has been contacted with an Agrobacterium strain harboring a vector capable of transferring a gene to a plant cell. In a particular embodiment, the plant tissue is transformed callus. In some embodiments, the plant tissue is derived from a tree selected from the group consisting of: Eucalyptus, pine, Populus, and sweetgum. In illustrative embodiments, the plant tissue is derived from a Eucalyptus tree selected the group consisting of: E. grandis, E. urophylla, E. nitens, E. globulus, E. dunnii, E. saligna, E. occidentalis, E. camaldulensis, and hybrid crosses thereof. In other illustrative embodiments, the plant tissue is derived from a pine tree selected from the group consisting of: Eastern white pine, Western white, Sugar pine, Red pine, Pitch pine, Jack pine, Longleaf pine, Shortleaf pine, Loblolly pine, Slash pine, Virginia pine, Ponderosa pine, Jeffrey pine, Pond pine, and Lodgepole pine, Radiata pine, and hybrid crosses thereof.

In some embodiments, the concentration of meta-topolin in the medium is from about 0.01 to about 100 μM, or from about 0.1 to about 20 μM. In some embodiments, the medium comprises TDZ and the concentration of TDZ is from about 0.025 to about 0.1 μM. In some embodiments, the medium comprises 4-CPPU and the concentration of 4-CPPU is from about 0.025 to about 0.1 μM. In some embodiments, the medium further comprises Zeatin. In some embodiments, the medium further comprises an auxin. For example, the auxin may be selected from the group consisting of NAA, 2,4-D, IBA, and IAA. Typically, the medium comprises one or more ingredients selected from the group consisting of: salts, vitamins, glucose, sucrose and a gelling agent. In some embodiments, the plant tissue is incubated in the medium for at least 1 day or for at least one week.

In some embodiments, the methods further comprise transforming at least one cell of the plant tissue with a foreign DNA by exposing the plant tissue to an Agrobacterium strain containing a transformation vector carrying the foreign DNA, wherein the foreign DNA is transferred to at least one cell of the plant tissue. The methods may further comprise selecting for the transformed plant cell(s).

In another aspect, the disclosure describes a method for regenerating a Eucalyptus plant comprising: (a) providing a Eucalyptus explant and (b) incubating the Eucalyptus explant in a medium that comprises meta-topolin until one or more shoots or shoot primordia are formed. In some embodiments, the Eucalyptus explant is selected from the group consisting of: E. grandis, E. urophylla, E. nitens, E. globulus, E. dunnii, E. saligna, E. occidentalis, E. camaldulensis, and hybrid crosses thereof. For example, the Eucalyptus explant is an explant selected from the group consisting of: a leaf explant, a petiole explant, an internode explant, a floral tissue explant, or an embryogenic tissue explant. In some embodiments, the Eucalyptus explant is an explant that has been contacted with an Agrobacterium strain harboring a vector capable of transferring a gene to a plant cell. In some embodiments, the medium further comprises a cytokinin selected from the group consisting of 1-phenyl-3-(1,2,3-thiadiazol-5-yl)urea (thidiazuron, TDZ), N-(2-Chloro-4-pyridyl)-N′-phenylurea (4-CPPU), zeatin, and auxin.

### BRIEF DESCRIPTION OF THE DRAWINGS

FIG. 1 is a series of photographs of internode explants regenerated on H2_{—}2 medium containing 5 μM meta-topolin+0.1 μM 4-CPPU+0.025 μM TDZ.

FIG. 2 is a series of photographs of transformed internode explants stained with 5-bromo-4-chloro-3-indolyl-β-D-glucuronic acid (X-gluc) for β-glucuronidase (GUS) activity.

FIG. 3 is a photograph of leaf and shoot explants from putative transgenic shoots that were sampled and treated with x-gluc to detect the presence and expression of GUS gene.

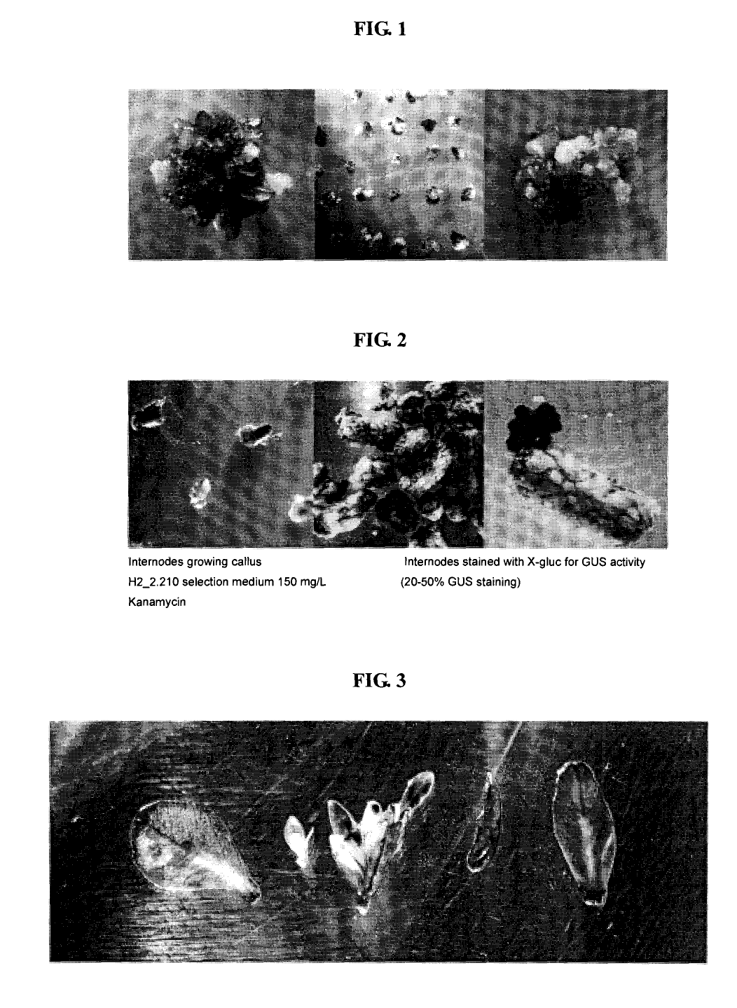

### DETAILED DESCRIPTION

The methods of the present disclosure provide for genotype-independent shoot regeneration to overcome the low regeneration efficiencies typically obtained from elite clones or genotypes of trees. In its broadest aspect, the methods relate to increasing the efficiencies of shoot regeneration from tree explants. Increases in shoot regeneration frequency are accomplished by culturing the explants on a medium that includes meta-topolin. The media can also be used to regenerate plants from any plant cell that has been transformed with one or more genes or selectable markers. The inventive media and methods are particularly useful for regenerating transgenic forest trees.

In the description that follows, a number of terms are utilized extensively. Unless defined otherwise, all technical and scientific terms used herein share the same meaning as commonly understood by one of ordinary skill in the art to which this invention belongs. The following definitions are provided to facilitate understanding of the invention. Units, prefixes, and symbols may be denoted in their accepted SI form.

Unless explicitly described to the contrary, the word “comprise” and variations such as “comprises” or “comprising”, and “having” and/or “including’ will be understood to include the information described in the body of the claim, for example, but not to exclude information not explicitly set forth.

The terms “a” and “an” as used herein mean “one or more” unless the singular is expressly specified.

As used herein, when referring to a numerical value, the term “about” refers to plus or minus 10% of the enumerated value, unless otherwise stated.

The term “Agrobacterium-mediated transformation” refers to a method by which DNA is stably inserted into the genome of a plant cell through the use of the Ti (tumor-inducing) plasmid from Agrobacterium tumefaciens. A small portion of the Ti plasmid, known as the T-DNA, is incorporated into the nucleus of the host plant cell. Alternatively, the Ri plasmid from Agrobacterium rhizogenes may be used for transformation. In Agrobacterium-mediated transformation, the gene(s) or DNA intended for plant introduction is positioned between the left and right borders of the T-DNA.

The term “auxin” encompasses a class of plant growth regulators that are characterized principally by their capacity to stimulate cell division in excised plant tissues. In addition to their role in cell division and cell elongation, auxins influence other developmental processes, including root initiation. In the present invention, auxin and auxin-type growth regulators include, but are not limited to, naphthaleneacetic acid (NAA), 2,4-Dichlorophenoxy acetic acid (2,4-D), indole-3-butyric acid (IBA), and indole-3-acetic acid (IAA).

The term “callus” refers to a dedifferentiated proliferating mass of cells or tissue.

A “cloning vector” is a genetic element, such as a plasmid, cosmid, or bacteriophage, that has the capability to replicate autonomously in a host cell. Cloning vectors typically comprise sites in which foreign DNA sequences can be inserted in a determinable orientation and a marker gene that encodes a product suitable for the identification and selection of cells transformed with the cloning vector. Marker genes include genes whose products confer antibiotic or herbicide resistance. The insertion of a foreign DNA sequence into a cloning vector does not interfere with an essential biological function of the cloning vector or the marker gene.

The term “cytokinin” refers to a class of plant growth regulators that are characterized by their ability to stimulate cell division and shoot organogenesis in tissue culture. In the present invention, cytokinins include, but are not limited to, N⁶-benzylaminopurine (BAP), N⁶-benzyladenine (BA), zeatin, kinetin, thiadiazuron (TDZ), meta-topolin, 2-isopentenyladenine (2ip), and 4-CPPU (N-(2-chloro-4-pyridyl)-N′-phenylurea)).

The term “effective amount” as used herein refers to the amount or concentration of a compound, such as a cytokinin, administered to a plant or explant such that the compound stimulates or invokes one or more of a variety of plant growth responses. A plant growth response includes, among others, the induction of stem elongation, the promotion of shoot or root formation, the stimulation of callus formation, enhancement of leaf growth, stimulation of seed germination, increase in the dry weight content of a number of plants and plant parts, and the like.

The term “elite genotype” refers to a commercially important plant (e.g., tree) genotype which has a known field performance and is obtained through breeding and selection. For the purposes of this application, the terms “genotype” and “clone” are used interchangeably.

The term “embryogenic tissue” refers to any tissue, derived from a plant, which is capable of producing one or more cotyledonary somatic embryos. For example, the term “embryogenic tissue” includes conifer embryogenic tissue.

The term “explant” refers to a plant part that is capable of being transformed and subsequently regenerated into a plant. Typical explants include explants derived from leaf, petiole, floral tissue, internodal tissues, immature embryos or embryogenic tissues, somatic embryos and their organs, somatic embryogenic callus, and cotyledons.

A “foreign DNA” is DNA isolated from another species or from the species of interest and re-introduced into the same species. The DNA may be a structural gene, an antisense gene, DNA fragments, etc.

A “gene” is a heritable DNA sequence that is transcribed into messenger RNA (mRNA) which is then translated into a sequence of amino acids characteristic of a polypeptide.

The term “operably linked” describes combining two or more molecules in such a fashion that in combination they function properly in a plant cell. For instance, a promoter is operably linked to a structural gene when the promoter controls transcription of the structural gene.

A “plant promoter” is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as leaves, roots, or seeds. Such promoters are referred to as tissue-preferred promoters. Promoters which initiate transcription only in certain tissue are referred to as tissue-specific promoters. A cell type-specific promoter primarily drives expression in certain cell types in one or more organs, for example, vascular cells in roots or leaves. An inducible or repressible promoter is a promoter which is under environmental control. Examples of environmental conditions that may effect transcription by inducible promoters include anaerobic conditions or the presence of light. Tissue specific, tissue preferred, cell type specific, and inducible promoters constitute the class of non-constitutive promoters. A constitutive promoter is a promoter which is active under most environmental conditions, and in most plant parts.

The term “pre-culture medium,” as used herein, is the nutrient medium upon which plant explants are cultured prior to transformation with Agrobacterium and is needed for increasing transformation efficiency and plant regeneration. The pre-culture medium may comprise an inducer of Agrobacterium, such as acetosyringone, and optionally, plant growth regulators, including auxin and cytokinin.

The term “shoot regeneration medium” refers to a medium designed to regenerate plant shoots of interest, e.g., transgenic shoots. The shoot regeneration medium comprises inorganic salts, a mixture of amino acids and vitamins, an antioxidant, organic nitrogen, and plant growth regulators. In various embodiments of the methods described herein, the shoot regeneration medium includes the cytokinin meta-topolin.

The term “structural gene” is a DNA sequence that is transcribed into messenger RNA (mRNA) which is then translated into a sequence of amino acids characteristic of a specific polypeptide.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “tree” refers to any perennial vegetation that accumulates a wood core. Trees include angiosperms and gymnosperm species. Examples of trees include poplar, Eucalyptus, Douglas fir, pine, sugar and Monterey, nut trees, e.g., walnut and almond, fruit trees, e.g., apple, plum, citrus and apricot, and hardwood trees, such as ash, birch, oak, and teak. Of particular interest for commercial purposes are conifers such as pine, fir, spruce, Eucalyptus, Acacia, aspen, Sweetgum, and poplar.

The present invention provides methods for regenerating shoots from transformed or untransformed tree explants. The methods contemplate culturing tree explants in the presence of N⁶-(meta-hydroxybenzyl)adenine (referred to herein as “meta-topolin”), and optionally, one or more additional cytokinins or growth factors. As shown herein, it was found that meta-topolin plays a key role in increasing the frequency of shoot regeneration in explants of elite tree species. Indeed, it was observed that regeneration efficiency is directly dependent on meta-topolin levels. These results indicate that there is a positive correlation between meta-topolin concentration in regeneration medium and overall regeneration efficiency.

Cytokinins are a class of N⁶ substituted purine derivative plant hormones that regulate cell division, as well as a large number of developmental events, such as plant growth, cell division, shoot initiation and development, root differentiation and development, leaf development, chloroplast development, and senescence (Mok et al. (1995) Cytokinins. Chemistry, Action and Function. CRC Press, Boca Raton, Fla., pp. 155-166). The highly active aromatic cytokinin, meta-topolin, was identified in extracts of mature poplar leaves (Strnad et al., Phytochemistry, 45: 213-218 (1997)). The present inventors discovered that the inclusion meta-topolin in a regeneration medium results in shoot regeneration of explants from tree species that otherwise exhibit low regeneration efficiency.

Accordingly, in one aspect the present invention provides a method for regenerating a plant from plant tissues comprising: (a) culturing plant tissue in a medium that comprises meta-topolin and (b) incubating the plant tissue until one or more shoots or shoot primordia formed. In some embodiments, the shoots are then elongated and rooted to become plants.

The present methods and compositions may be applied to a variety of tissues from plant species that are otherwise difficult to regenerate. In some embodiments, the plant tissue is derived from a tree selected from the group consisting of: Eucalyptus, pine, Populus, and sweetgum. In some embodiments, the methods and compositions may be applied to Eucalyptus shoot regeneration. Any Eucalyptus explant may be regenerated according to the methods of described herein, including Eucalyptus trees grown in natural environments and Eucalyptus explants clonally propagated. The explant may be selected from any Eucalyptus species, including Eucalyptus alba, Eucalyptus bancroftii, Eucalyptus botryoides, Eucalyptus bridgesiana, Eucalyptus calophylla, Eucalyptus camaldulensis, Eucalyptus citriodora, Eucalyptus cladocalyx, Eucalyptus coccifera, Eucalyptus curtisii, Eucalyptus dalrympleana, Eucalyptus deglupta, Eucalyptus delagatensis, Eucalyptus diversicolor, Eucalyptus dunnii, Eucalyptus ficifolia, Eucalyptus globulus, Eucalyptus gomphocephala, Eucalyptus gunnii, Eucalyptus henryi, Eucalyptus laevopinea, Eucalyptus macarthurii, Eucalyptus macrorhyncha, Eucalyptus maculata, Eucalyptus marginata, Eucalyptus megacarpa, Eucalyptus melliodora, Eucalyptus nicholii, Eucalyptus nitens, Eucalyptus nova-angelica, Eucalyptus obliqua, Eucalyptus occidentalis, Eucalyptus obtusiflora, Eucalyptus oreades, Eucalyptus pauciflora, Eucalyptus polybractea, Eucalyptus regnans, Eucalyptus resinifera, Eucalyptus robusta, Eucalyptus rudis, Eucalyptus saligna, Eucalyptus sideroxylon, Eucalyptus stuartiana, Eucalyptus tereticornis, Eucalyptus torelliana, Eucalyptus urnigera, Eucalyptus urophylla, Eucalyptus viminalis, Eucalyptus viridis, Eucalyptus wandoo, Eucalyptus youmanni, and hybrids thereof.

In some embodiments, the methods provide for transforming clonally propagated tissues from pine. In some embodiments, the target plant is selected from the group consisting of Pinus banksiana, Pinus brutia, Pinus caribaea, Pinus clasusa, Pinus contorta, Pinus coulteri, Pinus echinata, Pinus eldarica, Pinus elliotii, Pinus jeffreyi, Pinus lambertiana, Pinus massoniana, Pinus monticola, Pinus nigra, Pinus palustris, Pinus pinaster, Pinus ponderosa, Pinus radiata, Pinus resinosa, Pinus rigida, Pinus serotina, Pinus strobus, Pinus sylvestris, Pinus taeda, Pinus virginiana, Abies amabilis, Abies balsamea, Abies concolor, Abies grandis, Abies lasiocarpa, Abies magnifica, Abies procera, Chamaecyparis lawsoniona, Chamaecyparis nootkatensis, Chamaecyparis thyoides, Juniperus virginiana, Larix decidua, Larix laricina, Larix leptolepis, Larix occidentalis, Larix siberica, Libocedrus decurrens, Picea abies, Picea engelmanni, Picea glauca, Picea mariana, Picea pungens, Picea rubens, Picea sitchensis, Pseudotsuga menziesii, Sequoia gigantea, Sequoia sempervirens, Taxodium distichum, Tsuga canadensis, Tsuga heterophylla, Tsuga mertensiana, Thuja occidentalis, and Thuja plicata.

In particular embodiments, the plant may be Eucalyptus grandis or its hybrids, Pinus radiata, Pinus taeda L. (loblolly pine) or its hybrids, Populus nigra, Populus deltoides, Populus alba, or Populus hybrids, Acacia mangium, or Liquidamber styraciflua.

In some embodiments, the methods contemplate the regeneration of Eucalyptus explants obtained from a stock culture of elite Eucalyptus genotypes. Micropropagated shoot cultures may be generated by harvesting newly flushed apical or axillary shoots and surface sterilizing the tissues in a sterilization solution. Sterilization solutions, such as 1-5% bleach solution, are known in the art and repeated rinsing with sterile, distilled water can be performed. Eucalyptus stock cultures can be maintained as shoot clusters on a maintenance medium comprising inorganic salts, carbon sources, vitamins, and cytokinins. For example, stock cultures may be maintained on Eucalyptus Maintenance (EM) medium (Table 1) comprising Woody Plant Medium (WPM) salts (Loyd and McCown, 1980) and N⁶-benzyladenine (BA). Alternatively, other salt media, such as MS medium (Murashige and Skoog 1962) or DKW medium (Driver, J. A.; Kuniyuki, A. H.1984. In vitro propagation of Paradox walnut rootstock, Juglans hindsii X Juglans regia, tissue culture. HortScience. 19:507-509), may be used.

TABLE 1
Eucalyptus Maintenance Medium (EM medium)

| Medium | Amount per Liter | |
|---|---|---|
| Components | of Medium | |
| WPM salts | 1 | package (Sigma) |
| Ca(NO₃)₂•4H₂O | 3.7 | g |
| MgSO₄•4H₂O | 0.37 | g |
| Nicotinic Acid | 0.5 | mg |
| Thiamine•HCl | 0.5 | mg |
| Pyridoxin•HCl | 0.5 | mg |
| D-Pantothenic Acid | 1.0 | mg |
| Myo-inositol | 0.1 | g |
| BA | 0.1-1 | mg |
| Bacto-agar | 5-8 | g |

The methods include the regeneration of explants independent of age or developmental stage. Explants may be derived from a variety of plant tissues including leaf, petiole, floral tissue, internodal tissues, immature embryos or embryogenic tissues, somatic embryos and their organs, and somatic embryogenic callus, and cotyledons. Tree explants obtained from the stock culture can be used for transformation. Tree explants may be selected from one or more of leaf, petiole, internodal, and floral tissues. In suitable embodiments, leaf explants are selected due to their abundant supply and ease of transformation. The tip portion of leaves may be removed or punctured with a forcep to increase the number of wounded cells. Leaf explants are typically placed on the medium abaxial side down.

The methods also include the regeneration of shoots from plant tissues that have been wounded. The terms “wounded” or “wounding” refers to the introduction of a wound in the plant tissue. Wounding of plant tissue may be achieved, for example, by punching, by using a blade, by maceration, by bombardment with microprojectiles, etc. Micro-particle bombardment may be conducted by any technique known to those skilled in the art. Such techniques include, but are not limited to, tungsten or gold micro-particle bombardment (without DNA). It is anticipated that wounding provides exposed tissue which is competent for transformation and regeneration.

The present invention teaches methods for shoot regeneration wherein explants are cultured on a medium comprising a mixture of amino acids and vitamins, plant growth regulators, glucose, and an antioxidant. In accordance with the methods described herein, meta-topolin may be added to any basal medium that is appropriate for the tree species being regenerated. Examples of minimal medium suitable for the growth of plant tissue include B5 medium (Gamborg et al., Exptl. Cell. Res., 50: 151-158 (1968)); MS medium (Murashige and Skoog, (1962), Physiologia Plantarium 15: 443-97) and N6 medium (Chu et al., Scientia Sinica, 18: 659-668 (1975). For example, the following minimal salts are typically found in MS minimal media: MgSO₄.7H₂O, CaCl₂.2H₂O, KNO₃, NH₄NO₃, KH₂PO₄, MgSO₄.4H₂O, ZnSO₄.7H₂O, CuSO₄.5H₂O, CoCl₂.6H₂O, K₁, H₃BO₃, Na₂MoO₄.2H₂O, FeSO₄.7H₂O, and Na₂EDTA. The basal media may also be a woody plant medium (WPM) as described in Lloyd and McCown, Proc. Int. Plant Prop. Soc. 30:421-437 (1980) supplemented with 2% sucrose and 650 mg/L calcium gluconate and 500 mg/L MES are added as pH buffers as described in De Block, Plant Physiol. 93:1110-1116 (1990).

The basal medium may include vitamins of B5 or of any other vitamin composition and a carbon source (e.g., sucrose or glucose 1 to 6% w/v). The media may further contain a gelling agent agar 0.6 to 1.2% or phytagel (gelrite) 0.2 to 0.5% w/v. The medium is typically adjusted to pH 5.4 to 6.2 prior to autoclaving.

As noted above, the regeneration media may include an auxin. For example, the auxin or an auxin-type growth regulator may be selected from the group consisting of naphthaleneacetic acid (NAA), 2,4-Dichlorophenoxyacetic acid (2,4-D), indole-3-butyric acid (IBA), and indole-3-acetic acid (IAA). The concentration range of said auxin is typically from about 0.1 to about 10 mg/l. The type auxin concentration is from about 0.2 to about 5 mg/l.

The regeneration media may include other growth regulators or plant hormones. Suitable growth regulators are 6-benzylamino (BAP), thidiazuron (TDZ) purine N-(2-Chloro-4-pyridyl)-N′-phenylurea (4-CPPU), kinetin, zeatin, and 6-benzyladenine. TDZ has several homologous compounds, some of which work as well as TDZ. Therefore, compounds which have the same effect as the itemized growth regulators are encompassed by this invention. TDZ not only acts as a growth regulator, but also has the advantage of inducing synthesis and/or accumulation of other growth regulators. Regeneration by somatic embryogenesis in many plants can be induced by TDZ during prolonged culture. It is likely that TDZ affects auxins as well as cytokinins. The use of BAP, 4-CPPU, or TDZ together or the use of an auxin and 4-CPPU or an auxin and TDZ are effective as well. In suitable embodiments, meta-topolin is combined with one or both of TDZ and 4-CPPU.

The regeneration medium consists of MS basal salts with modifications such as the replacement of sucrose with glucose and the mixture of agar and gelrite. Antibiotics, such as carbenicillin, cefotaxime, and timentin can also be included in the medium to prevent bacterial overgrowth after transformation. In a suitable embodiment, timentin is the antibiotic. The antibiotic concentration typically ranges from about 75 to 800 mg/l, typically about 250 mg/l. An exemplary regeneration medium for Eucalyptus is listed in Table 2.

TABLE 2
Eucalyptus Regeneration Medium

| Components for 1 Liter of | |
|---|---|
| Medium | Grams |
| KNO₃ | 1.9 |
| NH₄H₂PO₄ | 0.17 |
| MgSO₄•7H₂O | 0.37 |
| CaCl₂•2H₂O | 0.44 |
| FeSO₄•7H₂O | 0.0278 |
| Na₂EDTA•2H₂O | 0.0373 |
| MES (Duchefa m1501) | 600.0 |
| MnSO₄•H₂O | 0.0169 |
| ZnSO₄•7H₂O | 0.0086 |
| CuSO₄•5H₂O | 0.000025 |
| CoCl₂•6H₂O | 0.0000125 |
| KI | 0.00083 |
| H₃BO₃ | 0.0062 |
| Na₂MoO₄•2H₂O | 0.00025 |
| Glucose | 20.0 |
| Myo-inositol | 0.100 |
| Nicotinic Acid | 0.0005 |
| Thiamine | 0.0001 |
| Pyridoxine | 0.0005 |
| Glycine | 0.002 |
| Zeatin | 2.2 |
| Gelrite | 1.0 |
| Agar | 5.0 |

In some embodiments, regeneration cultures are incubated at temperature 20-40° C. in white fluorescent light (40-100 μmol/m² s) 16 h photoperiod, until shoots are formed. This is accompanied by swelling at the basal part of the explant. At this stage, shoots may be harvested and transferred to an elongation medium or rooting medium. The elongation medium is typically a hormone free basal medium. The rooting medium is typically a hormone free basal medium or medium supplemented with auxins. In general, the exact concentration of the salts can be varied within limits without departing from the invention.

As soon as the shoots are regenerated, the tissue may be transferred to the elongation medium and, whenever necessary, from 200-400 mg/L timentin, typically 250 mg/L timentin, may be used to control remnant Agrobacterium. Timentin can be omitted from the culture media if the Agrobacterium is not present. Green and healthy shoots elongated to approximately 2-3 cm in length are excised and planted separately in the rooting medium.

Plant Transformation. In some embodiments of the methods, shoots are regenerated from explants that have been transformed. The two processes of transformation and regeneration are complementary. The complementarity of the two processes is such that the tissues which are successfully genetically transformed by the transformation process must be of a type and character, and must be in sufficient health, competency and vitality, so that they can be successfully regenerated into whole plants. Successful transformation and regeneration techniques have been demonstrated for monocots and dicots.

The most common methodology used for the transformation of cells of dicot plant species involves the use of the plant pathogen Agrobacterium tumefaciens. Although Agrobacterium-mediated transformation has been achieved in some monocots, other methods of gene transfer have been more effective, e.g., the polyethylene glycol method, electroporation, direct injection, particle bombardment, etc., as described by Wu in Plant Biotechnology (1989) pp. 35-51, Butterworth Publishers, Stoneham, Mass. The present invention will be useful with any method of transformation that includes plant regeneration steps. In a specific embodiment, the invention envisions the genetic transformation of tissues in culture derived from leaf or stem explants. The transformed tissues can be induced to form plant tissue structures, which can be regenerated into whole plants.

The transformation and regeneration methods described herein can be used for introducing any foreign DNA into a tree species, e.g., a Eucalyptus or pine species. Using the methods described herein and known in the art, any foreign DNA can be stably integrated into a plant cell and transmitted to progeny. For example, a gene involved in lignin biosynthesis, floral development, cellulose synthesis, nutrient uptake and transport, disease resistance, or enhanced resistance to an environmental condition can be introduced into a plant cell by the instant methods.

The details of the construction of the vectors containing such foreign genes of interest are known to those skilled in the art of plant genetic engineering and do not differ in kind from those practices which have previously been demonstrated to be effective in tobacco, petunia and other model plant species. The foreign gene should be selected as a marker gene (Jefferson et al. (1987) EMBO J. 6:3901-3907) or to accomplish some desirable effect in plant cells. This effect may be growth promotion, disease resistance, a change in plant morphology or plant product quality, or any other change which can be accomplished by genetic manipulation. The chimeric gene construction can code for the expression of one or more exogenous proteins, or can cause the transcription of negative strand RNAs to control or inhibit either a disease process or an undesirable endogenous plant function.

In some embodiments, the methods can be used for reducing gene expression in a tree species. Reduction of gene expression can be obtained by methods known in the art, including antisense suppression, co-suppression (sense suppression), and double stranded RNA interference. For a general review of gene suppression techniques, see Science, 288:1370-1372 (2000). Exemplary gene silencing methods are also provided in WO 99/49029 and WO 99/53050.

For antisense suppression, a cDNA sequence is arranged in a reverse orientation relative to the promoter sequence in a DNA construct. The cDNA sequence need not be full length relative to either the primary transcription product or fully processed mRNA. Generally, higher identity can be used to compensate for the use of a shorter sequence. Furthermore, the introduced sequence need not have the same intron or exon pattern, and identity of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of about 500 to about 3500 nucleotides is especially preferred. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other genes within a family of genes exhibiting identity or substantial identity to the target gene.

Another well known method of gene suppression in plants in sense co-suppression. Introduction of a nucleic acid sequence configure in the sense orientation provides an effective means by which to block the transcription of target genes. See, Assaad et al. Plant Mol. Bio. 22: 1067-1085 (1993); Flavell Proc. Natl. Acad. Sci. USA 91: 3490-3496 (1994); Stam et al. Annals Bot. 79: 3-12 (1997); Napoli et al., The Plant Cell 2:279-289 (1990); and U.S. Pat. Nos. 5,034,323, 5,231,020, and 5,283,184. For co-suppression, the introduced sequence need not be full length, relative to either the primary transcription product or fully processed mRNA. Suitably, the introduced sequence is not full length, so as to avoid a concurrent sense overexpression phenotype. In general, a higher identity in a shorter than full length sequence compensates for a longer, less identical sequence.

Using the methods of the instant invention, antisense suppression of a gene involved in lignin biosynthesis can be used to modify lignin content and/or composition in a transformed and regenerated Eucalyptus plant. It has been demonstrated that antisense expression of sequences encoding cinnamyl alcohol dehydrogenase (CAD) in poplar, N. tabacum, and pine leads to the production of lignin having a modified monomeric composition. Grand et al., Planta 163: 232-37 (1985), Yahiaoui et al., Phytochemistry 49: 295-306 (1998), and Baucher et al., Plant Physiol. 112: 1479 (1996), respectively. Accordingly, the methods of the present invention can be used to stably transform and regenerate Eucalyptus species having antisense suppression of a foreign DNA.

The methods of the present invention can be used for regulating floral development in a Eucalyptus or pine species. Several gene products have been identified as critical components for anther development and pollen formation. For example, premature degradation of callose, which is essential for the formation of the microspore cell wall and microspore release, is sufficient to cause male sterility. Worrall et al., Plant Cell 4:7:759-71 (1992). Accordingly, several methods have been developed for producing male sterile plants. U.S. Pat. No. 5,962,769, for example, describes transgenic plants rendered male sterile via expression of avidin. Avidin can be expressed constitutively, in a non-tissue specific manner, or in anther-specific tissues. In addition, male sterility can be induced by antisense suppression of chalcone synthase A gene. van der Meer et al., Plant Cell 4:253 (1992). By the methods of the present invention, male sterile Eucalyptus and pine species can be produced and regenerated.

In particular embodiments, Eucalyptus explant transformation is performed with different strains of A. tumefaciens harboring a transformation vector. The vector will typically carry a selectable marker gene operably linked to a promoter. Explants are incubated with the A. tumefaciens culture suspended in induction medium for 10-30 minutes. Alternatively, the explants may be transformed by vacuum infiltration, floral dip, and other methods of Agrobacterium-mediated transformation that are well known in the art. For a review of Agrobacterium-mediated transformation, see Gelvin, S B. Microbiol. Mol Biol Rev 67:1:16-37 (2003). Upon introduction of Agrobacterium, the explants are co-cultivated with the Agrobacterium for about 3 days on the co-culture medium. Following co-cultivation, the explants are transferred to a shoot regeneration medium for the recovery of transgenic shoots.

A 4-day recovery period is typically provided before the selection of transformed explants. To select for transformed explants, any selectable marker is added to the regeneration medium. Selectable markers include herbicides and antibiotics. Additionally, any screenable marker may be used to select a transformed plant. Examples of screenable markers include β-glucuronidase (GUS), green fluorescent protein (GFP), and luciferase. An herbicide selection agent may be used. Herbicides include Ally, (just, and Liberty. The herbicide concentration may vary depending on the sensitivity of the explants from a specific species. The selected explants are subcultured every two to three weeks until the formation of adventitious buds. Transformed adventitious shoots are separated from shoot clumps and are examined for transgene expression. Jefferson et al. EMBO: 6:13:901-3907 (1987). A reporter gene assay, such as GUS, may be used to determine transformation efficiency and to ensure that the transformed shoots are not escapes or chimeras.

Upon confirmation of transformation via expression of a screenable marker gene, Southern blot analysis, PCR analysis, or other method known in the art, the transformed shoots are preferably transferred to a medium for shoot elongation. The present invention contemplates a shoot elongation medium comprising MS salts, sucrose, auxin, and giberellic acid. NAA is a suitable auxin and GA3 is a suitable giberellic acid. The shoots are cultured on the shoot elongation medium for about 10 to about 14 days, preferably under low light conditions. For the elongation of E. dunii clones, additional auxin needs to be added to the elongation media and the shoots should be cultured in the dark for 2-4 weeks or under low light condition for the duration of the elongation.

Following shoot elongation, the shoots are excised and transferred to a root induction medium, which may contain plant growth regulators, such as auxin. Shoot excision may be made at the node or immediately below the node. Suitably, the shoots are excised from a node near the shoot apex. One such rooting medium is comprised of BTM-1 nutrients (Chalupa 1988), activated carbon (MeadWestvaco, Nuchar), and an extra amount of CaCl₂. Alternatively, other low-salt media may be used for the root induction medium, such as Woody Plant Medium and MS.

Depending on the light conditions, the rooting media may contain growth regulators to induce root formation. For example, under dark conditions that induce etiolation and auxin production in the shoot apical meristem, it may not be necessary to include auxin in the rooting medium. Additionally, if the shoots are excised in the dark, it may not be necessary to limit the origin of excision to the nodal regions and/or the shoot apex. Alternatively, if the rooting step occurs under light, it may be necessary to include auxin in the rooting medium. Furthermore, if the rooting step occurs in the light, it is preferable to excise the shoots at a node near the shoot apical meristem.

Wood, Pulp and Paper Products. Another aspect of the invention provides methods of obtaining wood, wood pulp, paper, and oil from a plant transformed and regenerated by the methods of the present invention. Methods for transforming and selecting a transgenic plant are provided above and are known in the art. A transformed plant can be cultured or grown under any suitable conditions. For example, pine can be cultured and grown as described in U.S. Patent Application Publication No. 2002/0100083. Eucalyptus can be cultured and grown as in, for example, Rydelius, et al., Growing Eucalyptus for Pulp and Energy, presented at the Mechanization in Short Rotation, Intensive Culture Forestry Conference, Mobile, Ala., 1994. Wood, wood pulp, paper, and oil can be obtained from the plant by any means known in the art.

As noted above, the wood and wood pulp obtained in accordance with this invention may demonstrate improved characteristics including, but not limited to any one or more of lignin composition, lignin structure, wood composition, cellulose polymerization, fiber dimensions, ratio of fibers to other plant components, plant cell division, plant cell development, number of cells per unit area, cell size, cell shape, cell wall composition, rate of wood formation, aesthetic appearance of wood, formation of stem defects, rate of growth, rate of root formation ratio of root to branch vegetative development, leaf area index, and leaf shape include increased or decreased lignin content, increased accessibility of lignin to chemical treatments, improved reactivity of lignin, increased or decreased cellulose content increased dimensional stability, increased tensile strength, increased shear strength, increased compression strength, increased shock resistance, increased stiffness, increased or decreased hardness, decreased spirality, decreased shrinkage, and differences in weight, density, and specific gravity.

Phenotype can be assessed by any suitable means. The plants can be evaluated based on their general morphology. Transgenic plants can be observed with the naked eye, can be weighed and their height measured. The plant can be examined by isolating individual layers of plant tissue, namely phloem and cambium, which is further sectioned into meristematic cells, early expansion, late expansion, secondary wall formation, and late cell maturation. The plants also can be assessed using microscopic analysis or chemical analysis.

Microscopic analysis includes examining cell types, stage of development, and stain uptake by tissues and cells. Fiber morphology, such as fiber wall thickness and microfibril angle of wood pulp fibers can be observed using, for example, microscopic transmission ellipsometry. See Ye and Sundstrom, Tappi J., 80:181 (1997). Wood strength, density, and grain slope in wet wood and standing trees can be determined by measuring the visible and near infrared spectral data in conjunction with multivariate analysis. See, U.S. Patent Application Publication Nos. 2002/0107644 and 2002/0113212. Lumen size can be measured using scanning electron microscopy. Lignin structure and chemical properties can be observed using nuclear magnetic resonance spectroscopy as described in Marita et al., J. Chem. Soc., Perkin Trans. 12939 (2001). The biochemical characteristic of lignin, cellulose, carbohydrates and other plant extracts can be evaluated by any standard analytical method known including spectrophotometry, fluorescence spectroscopy, HPLC, mass spectroscopy, and tissue staining methods.

### EXAMPLES

The present invention is further illustrated by the following examples, which should not be construed as limiting in any way.

### Example 1

### Meta-Topolin Promotes Shoot Regeneration of Elite Eucalyptus

A critical component for transforming elite Eucalyptus is to regenerate shoots from cells. While shoot proliferation from existing buds are relatively easy for some elite Eucalyptus, the regeneration of shoots from cells originated from mature trees through differentiation is usually the limiting step. Under the currently available technologies for transforming Eucalyptus, transgenic plants can only be obtained when transgenic cells can regenerate or develop into plants. Thus, lack of regeneration or low regeneration prohibits the possibility of transforming the elite Eucalyptus since the regeneration is required for the selection and development of transgenic cells.

Conventionally, the regeneration has been achieved by using growth regulators such as BAP, zeatin, kinetin, 2ip, and TDZ. In one situation, 4-CPPU was used for regeneration. However, for certain elite lines, none of the mentioned growth regulators or combinations worked either alone or in combination. The methods described in this Example demonstrate a way to overcome the regeneration problem for elite Eucalyptus genotypes that are selected from breeding programs. In particular, the use of the growth regulator meta-topolin alone or in combination with 4-CPPU and/or TDZ promoted the regeneration of cells of elite trees that otherwise were difficult to regenerate.

Plant Materials and culture conditions. All elite clones were maintained as shoot clumps in Eucalyptus maintenance (EM) medium (Table 1) in magenta boxes or large disposable vessels and subcultured every 4-6 weeks. Shoot clumps were divided as needed and maintained as stock culture. Unless noted otherwise, all cultures were grown under shaded, cool fluorescent light with a light intensity of 30-40 gE/m²/s with a photoperiod of 16 hours, and a temperature of 21° C.

Although leaves, petioles, internodes, floral tissues, and embryogenic tissues can be used for transformation and subsequent regeneration, leaf explants were selected because leaves are abundant. For transformation, explants of E. grandis clones 6075 and 6084 were incubated with Agrobacterium and were placed on co-culture medium, which was the same as the regeneration medium (Table 2) except with the following modifications: the addition of Agrobacterium inducer acetosyringone and the addition of plant growth regulators that are enriched in auxin. MS medium (Murashige and Skoog 1962) was used in the present co-culture medium; however, other salt media, such as Woody Plant Medium (WPM) salts (Loyd and McCown, 1980) or Lepoivre medium, may be used. Also, other Agrobacterium inducers may be used. Plant explants were co-cultured for four days under low light conditions or in the dark on the co-culture medium.

To test the ability of various cytokinins to stimulate adventitious regeneration, the effects of 4-CPPU, Zeatin, TDZ, meta-topolin, and adenine sulfate were compared. The levels of cytokinins varied from 0.025 to 10 μM. The base medium for this matrix is named H2_{—}2.0 (or H2_{—}2 for short), which is the primary MS-based regeneration medium (Table 2). The medium has been effective to induce shoot regeneration from many Eucalyptus species including E. grandis, E. dunnii, E. camaldulensis, E. urophylla, E. occidentalis, and E. saligna. The antibiotic timentin was added at 250 mg/L final concentration. Shoot regeneration was measured as the frequency of explants produced shoots. Explants were cultured on the regeneration medium for about 8-10 weeks before collection of final regeneration data.

In a first experiment, combinations of meta-topolin and TDZ were included in the regeneration media and the percent shoots was measured. Tables 3 and 4 shows the matrix of tested combinations and the results for clones 6084 and 6075, respectively.

TABLE 3
Meta-Topolin Matrix with TDZ for Clone 6084.

| | Cytokinins | | | | | | | |
|---|---|---|---|---|---|---|---|---|
| | TDZ | | | Zeatin | Meta-Topolin | | | |
| | Concentration in μM | | | | | | | |
| Treatment | 0.025 | 0.05 | 0.1 | 2.2 | 0.1 | 2.5 | 5.0 | % Shoots |
| H2_2.0 | | | | X | | | | 2.0% |
| H2_2.13 | | | | | X | | | 0.0% |
| H2_2.14 | | | | | | X | | 15.0% |
| H2_2.15 | | | | | | | X | 9.0% |
| H2_2.16 | X | | | | X | | | 5.0% |
| H2_2.17 | | X | | | X | | | 0.0% |
| H2_2.18 | | | X | | X | | | 1.0% |
| H2_2.19 | X | | | | | X | | 14.0% |
| H2_2.20 | | X | | | | X | | 24.2% |
| H2_2.21 | | | X | | | X | | 8.9% |
| H2_2.22 | X | | | | | | X | 18.2% |
| H2_2.23 | | X | | | | | X | 11.9% |
| H2_2.24 | | | X | | | | X | 20.0% |

TABLE 4
Meta-Topolin Matrix with TDZ for Clone 6075

| | Cytokinins | | | | | | | |
|---|---|---|---|---|---|---|---|---|
| | TDZ | | | Zeatin | Meta-Topolin | | | |
| | Concentration in μM | | | | | | | |
| Treatment | 0.025 | 0.05 | 0.1 | 2.2 | 0.1 | 2.5 | 5.0 | % Shoots |
| H2_2.0 | | | | X | | | | 2.4% |
| H2_2.13 | | | | | X | | | 1.3% |
| H2_2.14 | | | | | | X | | 3.7% |
| H2_2.15 | | | | | | | X | 12.1% |
| H2_2.16 | X | | | | X | | | 0.0% |
| H2_2.17 | | X | | | X | | | 4.9% |
| H2_2.18 | | | X | | X | | | 1.2% |
| H2_2.19 | X | | | | | X | | 4.8% |
| H2_2.20 | | X | | | | X | | 15.7% |
| H2_2.21 | | | X | | | X | | 18.1% |
| H2_2.22 | X | | | | | | X | 29.8% |
| H2_2.23 | | X | | | | | X | 33.7% |
| H2_2.24 | | | X | | | | X | 34.9% |

The data indicate that the primary Eucalyptus regeneration medium H2_{—}2 failed to induce high rate of regeneration from these elite clones. Similarly, regeneration media having less than 0.1 μM meta-topolin was insufficient to promote a shoot formation of greater than 5%. Thus, these clones are very difficult to regenerate. The data further indicate that concentrations of meta-topolin of at least 2.5 μM result in a significant increase in adventitious shoot formation for both clones 6084 and 6075. This effect can be enhanced when TDZ is further included in the regeneration media. There is a synergy from adding both meta-topolin and TDZ, but the increase in meta-topolin contributes a far greater increase in shoot regeneration. For example, at any TDZ level, the improvement in regeneration as the increase in meta-topolin is more than 10% for nearly all increments of the meta-topolin (as high as 25%). However, the improvement in regeneration as the increase in TDZ is mostly less than 5% (only one incidence reached 10.9% when meta-topolin was at 2.5 μM). As such, regeneration media containing meta-topolin can be used in methods to enhance shoot formation of elite Eucalyptus species.

Since the improvement in regeneration was contributed by the increase of meta-topolin treatment from 0.1 to 5.0 μM, the concentration range was extended to 20 μM in the next experiment. Table 5 shows the matrix of tested combinations and the results for clones 6084 and 6075. The data indicate that a meta-topolin concentration from about 2.5 to about 10 μM resulted in significantly higher shoot regeneration frequency for clones 6075 and 6084. Also, it shows that TDZ alone, even at the relatively high concentration at 0.1 μM, was not effective in promotion of shoot regeneration.

TABLE 5
Meta-Topolin × TDZ matrix for Clones 6075 and 6084.

| | | | Percentage of Adventitious | |
|---|---|---|---|---|
| | | | Shoots Formed | |
| Treatment | Meta-Topolin (μM) | TDZ (μM) | A-6075 | A-6084 |
| H2_2.20 | 2.5 | 0.05 | 48.5 | 40% |
| H2_2.23 | 5 | | 52% | 46.2% |
| H2_2.137 | 10 | | 41% | 47.5% |
| H2_2.138 | 20 | | 3% | 4.9% |
| H2_2.113 | 0 | 0.1 | 3% | 0% |
| H2_2.21 | 2.5 | | 23.2% | 16.2% |
| H2_2.24 | 5 | | 44% | 28.2% |
| H2_2.140 | 10 | | 19.2% | 21% |
| H2_2.141 | 20 | | 0% | 0% |

Combinations of meta-topolin and 4-CPPU were included in the regeneration media and the percent shoot formation was measured. Table 6 shows the matrix of tested combinations and the results for clones 6075 and 6084. The data indicate that effective meta-topolin concentration at 2.5 or 5 μM resulted in high frequency of adventitious shoots regardless of the 4-CPPU concentrations for clones 6075 and 6084. Lower concentrations of 4-CPPU are not effective in induce adventitious shoot formation. Only high concentration at 0.1 μM was effective, and there was additive enhancement in shoot regeneration when 4-CPPU was together with meta-topolin.

TABLE 6
Meta-Topolin with 4-CPPU for Regeneration of Clones 6075 and 6084.

| | Meta-Topolin | | 4-CPPU | | | % Shoot | |
|---|---|---|---|---|---|---|---|
| | μM | | | | | Regeneration | |
| Media Code: | 2.5 | 5 | 0.025 | 0.05 | 0.1 | A-6075 | A-6084 |
| H2_2.25 | | | | | X | 25.64% | 42.67% |
| H2_2.14 | X | | | | | 28.75% | 40.00% |
| H2_2.202 | X | | | | X | 50.00% | 73.33% |
| H2_2.15 | | X | | | | 42.31% | 41.33% |
| H2_2.203 | | X | | | X | 60.26% | 60.00% |
| H2_2.213 | | | | X | | 5.00% | 1.33% |
| H2_2.214 | X | | | X | | 42.68% | 69.33% |
| H2_2.215 | | X | | X | | 57.83% | 41.56% |
| H2_2.216 | | | X | | | 1.33% | 0.00% |
| H2_2.217 | X | | X | | | 38.16% | 36.00% |
| H2_2.218 | | X | X | | | 57.33% | 22.78% |

Combinations of meta-topolin, 4-CPPU, zeatin, and auxin were included in the regeneration media and the percent shoot regeneration was measured. Table 7 shows the matrix of tested combinations and the results for clones 6084 and 6075. The data indicate that the treatments with meta-topolin had relatively high shoot regeneration frequencies for clones 6075 and 6084. Combinations of 4-CPPU at 0.025, 0.05 or 0.1 μM and zeatin at 10 μM and NAA at 0.53 μM were not effective in shoot induction. In general, the presence of 10 μM zeatin and 0.53 μM NAA resulted in relatively low regeneration when used with the effective concentrations of meta-topolin and/or 4-CPPU.

TABLE 7
Meta-Topolin with 4-CPPU, Zeatin, and NAA.

| | Meta- | | | | | | | | |
|---|---|---|---|---|---|---|---|---|---|
| | Topolin | | 4-CPPU | | | Zeatin | NAA | % Shoot | |
| | μM | | | | | | | Regeneration | |
| Media Code: | 2.5 | 5 | 0.025 | 0.05 | 0.1 | 10 | 0.53 | A-6075 | A-6084 |
| H2_2.45 | | | | | X | X | X | 5.33% | 7.69% |
| H2_2.219 | | | | X | | X | X | 5.26% | 2.60% |
| H2_2.220 | | | X | | | X | X | 6.67% | 2.56% |
| H2_2.207 | X | | | | X | X | X | 16.00% | 15.58% |
| H2_2.221 | X | | | X | | X | X | 13.33% | 7.59% |
| H2_2.222 | X | | X | | | X | X | 21.33% | 7.69% |
| H2_2.208 | | X | | | X | X | X | 22.08% | 2.63% |
| H2_2.223 | | X | | X | | X | X | 21.05% | 10.26% |

Combinations of meta-topolin, 4-CPPU, and TDZ were further evaluated in a regeneration test and the percent of shoot regeneration was measured. Table 8 shows the matrix of tested combinations and the results for clones 6084 and 6075. The data indicate that meta-topolin concentration from of at least 2.5 μM resulted in significantly higher adventitious shoot regeneration for clones 6075 and 6084. The combination of 4-CPPU and/or TDZ enhanced the effect of the meta-topolin, but the combination of 4-CPPU and TDZ without meta-topolin was not nearly as effective. As such, regeneration media containing meta-topolin can be used in methods to enhance shoot formation of elite Eucalyptus clones.

TABLE 8
Meta-Topolin, 4-CPPU and TDZ for Regeneration of
Clones 6075 and 6084.

| Comparison For Regeneration | | | | | | | | | |
|---|---|---|---|---|---|---|---|---|---|
| | Meta- | | | | | | | | |
| | Topolin | | 4-CPPU | | | TDZ | | % Shoot | |
| Media | μM | | | | | | | Regeneration | |
| Code: | 2.5 | 5 | 0.025 | 0.05 | 0.1 | 0.025 | 0.05 | A-6075 | A-6084 |
| H2_2.182 | | | | | X | | X | 10.0% | 16.0% |
| H2_2.202 | X | | | | X | | | 48.0% | 50.0% |
| H2_2.203 | | X | | | X | | | 66.0% | 50.0% |
| H2_2.210 | | X | | | X | X | | 50.0% | 28.0% |
| H2_2.214 | X | | | X | | | | 56.0% | 62.0% |
| H2_2.215 | | X | | X | | | | 84.0% | 42.0% |
| H2_2.218 | | X | X | | | | | 56.0% | 46.0% |

Combinations of meta-mopolin, 4-CPPU, and TDZ that gave the best regeneration across multiple experiments were combined with two standard regeneration media that have worked with other clones in the past to become a regeneration screening matrix for new clonal material. Table 9 shows the matrix of tested combinations and the results for three elite clones: E. urophylla X grandis S-519, E. grandis clone S-619 (both from Suzano) and E. amplifolia clone EA5490 (from university of Florida). The data indicate that meta-topolin concentration from of at least 2.5 μM resulted in significantly higher adventitious shoot regeneration for clones S519 and EA5490. EA5490 showed the best regeneration with meta-topolin concentrations of 5 μM.

TABLE 9
Regeneration Screening Matrix

| | Meta- | | | | | | | | | | | |
|---|---|---|---|---|---|---|---|---|---|---|---|---|
| | Topolin | | 4-CPPU | | | Zeatin | NAA | TDZ | | % Shoot | | |
| Media | μM | | | | | | | | | Regeneration | | |
| Code: | 2.5 | 5 | 0.025 | 0.05 | 0.1 | 10 | 1 | 0.05 | 0.025 | S-519 | S-619 | EA5490 |
| H2_2 | | | | | | X | | | | 4.3% | 60.0% | 9.0% |
| H2_19 | | | | | | X | X | X | | 6.5% | 86.4% | 1.0% |
| H2_2.25 | | | | | X | | | | | 5.8% | 40.8% | 10.0% |
| H2_2.182 | | | | | X | | | X | | 15.2% | 91.2% | 1.0% |
| H2_2.202 | X | | | | X | | | | | 39.1% | 87.2% | 11.0% |
| H2_2.203 | | X | | | X | | | | | 42.8% | 80.8% | 20.0% |
| H2_2.210 | | X | | | X | | | | X | 46.4% | 89.6% | 19.0% |
| H2_2.214 | X | | | X | | | | | | 25.4% | 94.4% | 13.0% |
| H2_2.215 | | X | | X | | | | | | 29.7% | 92.8% | 17.0% |
| H2_2.218 | | X | X | | | | | | | 33.3% | 82.4% | 15.0% |

Table 10 shows shoot regeneration results of various media for clones of E. benthamii (Ben 83), E. amplifolia (5481 and 4810). Table 11 shows shoot regeneration results of various media for elite clones of E. urophylla X grandis (IPB-8, IPB-13, A11420, A6466, S6021, S1048, S1002). The composition of the media was the same as in Table 9. The data indicate that a meta-topolin concentration from at least 2.5 μM resulted in significantly higher adventitious shoot regeneration for many Eucalyptus clones.

TABLE 10
Shoot regeneration for E. benthamii and E. amplifolia

| Media | % Shoot Regeneration | | |
|---|---|---|---|
| Code: | Ben83 | 5481 | 4810 |
| H2_2 | 0.0% | 1.5% | 13.0% |
| H2_19 | 0.0% | 0.0% | 0.5% |
| H2_2.25 | 0.0% | 0.0% | 0.5% |
| H2_2.182 | 0.0% | 0.5% | 0.7% |
| H2_2.202 | 3.0% | 8.0% | 20.5% |
| H2_2.203 | 0.0% | 6.0% | 20.0% |
| H2_2.210 | 0.0% | 4.5% | 10.5% |
| H2_2.214 | 0.0% | 7.5% | 2.0% |
| H2_2.215 | 0.0% | 6.0% | 11.5% |
| H2_2.218 | 0.0% | 5.5% | 3.0% |

TABLE 11
Shoot regeneration for E. urophylla X grandis

| Media | % Shoot Regeneration | | | | | | | |
|---|---|---|---|---|---|---|---|---|
| Code: | IPB-7 | IPB-8 | IPB-13 | A11420 | A6466 | S6021 | S1048 | S1002 |
| H2_2 | 7.0% | 14.67% | 0.4% | 0.0% | 0.0% | 2.00% | 39.5% | 1.00% |
| H2_19 | 2.0% | 6.00% | 0.8% | 3.0% | 1.3% | 1.50% | 48% | 0.50% |
| H2_2.25 | 6.5% | 12.00% | 0.4% | 9.3% | 3.2% | 7.00% | 33% | 0.50% |
| H2_2.182 | 1.0% | 23.33% | 0.0% | 6.7% | 0.5% | 1.00% | 63% | 0.00% |
| H2_2.202 | 15.5% | 37.33% | 9.2% | 14.0% | 6.7% | 12.50% | 60% | 0.00% |
| H2_2.203 | 13.5% | 21.33% | 2.4% | 16.7% | 12.5% | 8.50% | 48.5% | 0.00% |
| H2_2.210 | 10.5% | 26.00% | 3.6% | 6.0% | 6.0% | 4.50% | 64% | 1.00% |
| H2_2.214 | 15.0% | 22.67% | 4.4% | 5.0% | 11.3% | 10.50% | 57.5% | 1.50% |
| H2_2.215 | 8.0% | 25.33% | 0.8% | 14.0% | 14.0% | 12.50% | 34.5% | 2.00% |
| H2_2.218 | 3.0% | 20.67% | 0.8% | 8.7% | 11.5% | 10.50% | 31% | 1.00% |

### Example 2

### Recovery of Stable Transgenic Calli from Regeneration/Selection Medium Containing Meta-Topolin, TDZ, and 4-CPPU for Clone 6084

The Agrobacterium strain EHA105 (Hood et al. 1993 Transgenic Res. 2:208-218) harboring the construct pWVR31 was used for the transformation. The pWVR31 construct contains the intron-containing GUS gene driven by ubiquitin 11 promoter and the neomycin phosphotransferase II gene (NPTII) under the control of ubiquitin 10 promoter.

Eucalyptus stock cultures maintained on Euc Maintenance medium were used as the sources of the explants. Internodes of elongated shoots were harvested and cut into sections of about 5 mm. For Agrobacterium preparation, a single colony of Agrobacterium containing pWVR31 from the plate was grown in 50 ml liquid YEP medium (10 g/l yeast extract, 10 g/l peptone and 50 mg/l NaCl, pH 7.0-7.2) with kanamycin at 100 mg/l and rifampicin 50 mg/l. Cultures were incubated overnight in a shaking incubator at 28° C. and 150 rpm. The over night culture with an OD of about 0.6 was spun down in a desktop centrifuge at 3000 g for 20 minutes and resuspended with 50 ml of Agrobacterium Induction Medium or AIM (WPM salts with glucose at 5 g/l, 250 μM acetosyringone, 2 mM phosphate buffer, and 0.05 M MES, pH 5.8). Explants were soaked in the Agrobacterium for 30-40 minutes before they were placed on co-cultivation medium for three days in a growth chamber with dim light.

After co-cultivation, explants were placed on selection medium, which was the regeneration medium H.210 (MS with 5 μM meta-topolin, 0.1 μM 4-CPPU and 0.025 μM TDZ) with 250 mg/l timentin and 100 mg/l kanamycin. At four weeks, the explants were transferred to the same medium with 150 mg/l kanamycin. The explants were subcultured regularly every 4 weeks until callus and shoot primordial formed. To determine GUS activity in putative transgenic lines, the explants were incubated in a substrate comprising 100 mM phosphate buffer (pH 7.0), 0.05% dimethyl suphoxide, 0.05% Triton X-100, 10 mM EDTA, 0.5 mM potassium ferrocyanide, and 1.5 mg/ml 5-bromo-4-chloro-3-indolyl-β-D-glucuronide (X-gluc). The explants were subjected to 10 min of vacuum before an overnight incubation at 37° C. Explants treated with X-gluc substrate were destained with 70% ethanol. The number of explants with blue callus was recorded for each treatment. The data is summarized in the following table and GUS positive calli developed on explants can be seen in FIG. 2.

TABLE 12
Recovery of Transgenic Calli

| | | | # Explants | |
|---|---|---|---|---|
| | | # | with GUS + | % Explants Produced |
| Clone | Operator | Explants | Callus | GUS + Calli |
| 6084 | 1 | 106 | 53 | 50.00% |
| 6084 | 2 | 50 | 10 | 20.00% |

### Example 3

### Recovery of Transgenic Plants from Regeneration/Selection Medium Containing Meta-Topolin, TDZ, and 4-CPPU for Clone 6075

This example describes the successful transformation and regeneration of a recalcitrant Eucalyptus clone 6075. Eucalyptus clone 6075 was transformed with a foreign DNA and then regenerated in media containing meta-topolin. In this example, clone 6075 was transformed with GUS marker gene construct pARB1001. This construct contains SUBIN::GUSIN::NOSTER reporter cassette and the neomycin phosphotransferase II gene (NPTII) under the ubiquitin 10 promoter. SUBIN indicates a ubiquitin promoter from P. radiata (See U.S. Pat. No. 6,380,459), which included genomic DNA coding the 5′-UTR and an intron.

The transformation procedure was similar to the previous example except that the Agrobacterium strain GV2260 (McBride and Summerfelt 1990 Plant Mol. Bio. 14:269-276) harboring the construct pWVR1001 was used for the transformation. Eucalyptus stock cultures maintained on Euc Maintenance medium were used as the sources of the explants. Explants coated with Agrobacterium were placed on co-cultivation medium for six days in a growth chamber with dim light. After co-cultivation, explants were placed on selection medium, which was the regeneration medium H.215 (MS with 5 μM meta-topolin and 0.05 μM 4-CPPU) with 250 mg/l timentin and 10 mg/l geneticin. The explants were transferred to fresh medium after two weeks. At four weeks, explants were transferred to the same medium except that the geneticin concentration was 15 mg/l. Next, the explants were transferred on the same medium every four weeks. Leaf and shoot explants from putative transgenic shoots were sampled and treated with x-gluc to detect the presence and expression of GUS gene. Transgenic lines showed strong GUS expression (FIG. 3). Three positive events were confirmed by PCR from 950 explants, resulting in a transformation percentage of (0.3%).

Transgenic shoots were bulked up on the propagation medium, which was the same as the stock medium. Elongation and rooting were following the same procedures as described above. This procedure resulted in transgenic plants for clone 6075 for the first time. As such, these data demonstrate that regeneration media containing meta-topolin can be used in methods to enhance shoot formation of elite Eucalyptus clones following transformation.

### Example 4

### Recovery of Transgenic Plants from Regeneration/Selection Medium Containing Meta-Topolin, TDZ, and 4-CPPU for Clone 6084

This example describes the successful transformation and regeneration of a recalcitrant Eucalyptus clone 6084. Eucalyptus clones can be transformed with a foreign DNA and then regenerated in media containing meta-topolin. For example, elite Eucalyptus clones can be transformed with a gene that encodes an enzyme involved in cellulose synthesis, such as a cellulose synthase. Cellulose synthase binds UDP-glucose and transfer the sugar to the non-reducing end of the nascent glucan chain. Using the methods of the present invention, a foreign DNA can be expressed in a transgenic plant.

The transformation procedure was similar to the previous example. Agrobacterium strain GV2260 harboring the construct pWV184 was used for the transformation. The construct contains the gene coding for the UDPG binding domain of the cellulose synthase driven by a xylem-specific promoter from loblolly pine and neomycin phosphotransferase II gene (NPTII) under the ubiquitin 10 promoter.

Eucalyptus stock cultures maintained on Euc Maintenance medium were used as the sources of the explants. Explants coated with Agrobacterium were placed on co-cultivation medium for five days in a growth chamber with dim light. After co-cultivation, explants were placed on selection medium, which was the regeneration medium H.215 (MS with 5 μM meta-topolin and 0.05 μM 4CPPU) with 250 mg/l timentin and 15 mg/l geneticin. The explants were transferred on the same medium every 4 weeks until shoot regeneration occurred. Shoot clumps were broken into individual shoots to get the best exposure to selection. Putative transgenic shoots were sampled for real time PCR (Polymerase Chain Reaction) analysis to detect the presence of the transgene. Three positive events were confirmed by PCR from 950 explants, resulting in a transformation percentage of (0.3%). Shoots with positive confirmation by RT-PCR were bulked up on the propagation medium, which was the same as the stock medium. Elongation and rooting were following the same procedures as described above. This experiment produced transgenic plants for clone 6084 for the first time. It is also the first time that a growth gene construct was introduced into this clone.

These data demonstrate that the methods of the present invention, including culture in a medium containing meta-topolin, are useful for the regeneration and transformation from otherwise recalcitrant clones.

### Example 5

### Meta-Topolin Promotes Shoot Regeneration of Elite Pine Species (Prophetic)

Explants are harvested from stock cultures of commercial pine trees and are cultured on the pre-culture medium as described in Example 2. To test the ability of various cytokinins to stimulate adventitious regeneration of loblolly pine, the effects of 4-CPPU, Zeatin, TDZ, meta-topolin, and adenine sulfate are compared. The levels of cytokinins varied from 0.025 to 10 μM. The base medium for this matrix is H2_{—}2 which contains the antibiotic timentin at 250 mg/L final concentration. Explants are placed in the regeneration medium and shoot regeneration is measured as the frequency of explants produced shoots. A comparison between treatments in which the culture media contained meta-topolin and treatments lacking meta-topolin indicate the effect of this compound to promote shoot regeneration of an elite pine species.

### Example 6

### Meta-Topolin Promotes Shoot Regeneration of Populus deltoides

Explants were harvested from stock cultures of commercial Populus trees (WV94) and were cultured on the pre-culture medium as described in Example 2. To test the ability of various cytokinins to stimulate adventitious regeneration of Populus, the effects of 4-CPPU, Zeatin, TDZ, meta-topolin, and adenine sulfate were compared. The levels of cytokinins varied from 0.025 to 10 μM. The base medium for this matrix is H2_{—}2 which contains the antibiotic timentin at 250 mg/L final concentration. Explants were placed in the regeneration medium and shoot regeneration was measured as the frequency of explants produced shoots. A comparison between treatments in which the culture media contained meta-topolin and treatments lacking meta-topolin indicate the effect of this compound to promote shoot regeneration of species of Populus. The results are shown in Table 13.

TABLE 13
Table 11. Shoot regeneration for Populus deltoides

| | Meta- | | | | | | | | | |
|---|---|---|---|---|---|---|---|---|---|---|
| | Topolin | | 4-CPPU | | | Zeatin | NAA | TDZ | | % Shoot |
| Media | μM | | | | | | | | | Primodia |
| Code: | 2.5 | 5 | 0.025 | 0.05 | 0.1 | 10 | 1 | 0.05 | 0.025 | WV94 |
| H2_2 | | | | | | X | | | | 7% |
| H2_19 | | | | | | X | X | X | | 7% |
| H2_2.25 | | | | | X | | | | | 33% |
| H2_2.182 | | | | | X | | | X | | 43% |
| H2_2.202 | X | | | | X | | | | | 50% |
| H2_2.203 | | X | | | X | | | | | 17% |
| H2_2.210 | | X | | | X | | | | X | 47% |
| H2_2.214 | X | | | X | | | | | | 37% |
| H2_2.215 | | X | | X | | | | | | 33% |
| H2_2.218 | | X | X | | | | | | | 27% |

### Example 7

### Meta-Topolin Promotes Shoot Regeneration of Sweetgum Species (Prophetic)

Explants are harvested from stock cultures of commercial sweetgum trees and are cultured on the pre-culture medium as described in Example 2. To test the ability of various cytokinins to stimulate adventitious regeneration of sweetgum, the effects of 4-CPPU, Zeatin, TDZ, meta-topolin, and adenine sulfate are compared. The levels of cytokinins varied from 0.025 to 10 μM. The base medium for this matrix is H2_{—}2 which contains the antibiotic timentin at 250 mg/L final concentration. Explants are placed in the regeneration medium and shoot regeneration is measured as the frequency of explants produced shoots. A comparison between treatments in which the culture media contained meta-topolin and treatments lacking meta-topolin indicate the effect of this compound to promote shoot regeneration of species of sweetgum.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can, of course, vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 units refers to groups having 1, 2, or 3 units. Similarly, a group having 1-5 units refers to groups having 1, 2, 3, 4, or 5 units, and so forth.

All publications, patent applications, patents, and other references mentioned herein are expressly incorporated by reference in their entirety, to the same extent as if each were incorporated by reference individually.

What is claimed is:

1. A method for regenerating a plant from plant tissues comprising:
(a) culturing plant tissue in a medium that comprises (i) meta-topolin and (ii) one or more additional cytokinins, wherein at least one cytokinin is selected from the group consisting of: thidiazuron (TDZ) and N-(2-Chloro-4-pyridyl)-N′-phenylurea (4-CPPU); and
(b) incubating the plant tissue until one or more shoots or shoot primordia are formed.

2. The method of claim 1, wherein the plant tissue is an explant selected from the group consisting of: a leaf explant, a petiole explant, an internode explant, a floral tissue explant, and an embryogenic tissue explant.

3. The method of claim 1, wherein the plant tissue is an explant that has been contacted with an Agrobacterium strain harboring a vector capable of transferring a gene to a plant cell.

4. The method of claim 1, wherein the plant tissue is transformed callus.

5. The method of claim 1, wherein the plant tissue is derived from a tree selected from the group consisting of: Eucalyptus, pine, Populus, and sweetgum.

6. The method of claim 4, wherein the plant tissue is derived from a Eucalyptus tree selected the group consisting of: E. grandis, E. urophylla, E. nitens, E. globulus, E. dunnii, E. saligna, E. occidentalis, E. camaldulensis, and hybrid crosses thereof.

7. The method of claim 5, wherein the plant tissue is derived from a pine tree selected from the group consisting of: Eastern white pine, Western white, Sugar pine, Red pine, Pitch pine, Jack pine, Longleaf pine, Shortleaf pine, Loblolly pine, Slash pine, Virginia pine, Ponderosa pine, Jeffrey pine, Pond pine, and Lodgepole pine, Radiata pine, and hybrid crosses thereof.

8. The method of claim 1, wherein the concentration of meta-topolin in the medium is from about 0.01 to about 100 μM.

9. The method of claim 8, wherein the concentration of meta-topolin in the medium is from about 0.1 to about 20 μM.

10. The method of claim 1, wherein the medium comprises TDZ and the concentration of TDZ is from about 0.025 to about 0.1 μM.

11. The method of claim 1, wherein the medium comprises 4-CPPU and the concentration of 4-CPPU is from about 0.025 to about 0.1 μM.

12. The method of claim 1, wherein the medium further comprises Zeatin.

13. The method of claim 1, wherein the medium further comprises auxin.

14. The method of claim 13, wherein the auxin is selected from the group consisting of NAA, 2,4-D, IBA, and IAA.

15. The method of claim 1, wherein the medium comprises one or more ingredients selected from the group consisting of: salts, vitamins, glucose, sucrose and a gelling agent.

16. The method of claim 1, wherein the plant tissue is incubated in the medium for at least 1 day.

17. The method of claim 1, wherein the plant tissue is incubated in the medium for at least 1 week.

18. The method of claim 1 further comprising, prior to step (a), transforming at least one cell of the plant tissue with a foreign DNA by exposing the plant tissue to an Agrobacterium strain containing a transformation vector carrying the foreign DNA, wherein the foreign DNA is transferred to at least one cell of the plant tissue.

19. The method of claim 18 further comprising selecting for the at least one transformed plant cell.

20. A method for regenerating a Eucalyptus plant comprising:
(a) providing a Eucalyptus explant and
(b) incubating the Eucalyptus explant in a medium that comprises meta-topolin until one or more shoots or shoot primordia are formed.

21. The method of claim 20, wherein the Eucalyptus explant is selected from the group consisting of: E. grandis, E. urophylla, E. nitens, E. globulus, E. dunnii, E. saligna, E. occidentalis, E. camaldulensis, and hybrid crosses thereof.

22. The method of claim 20, wherein the medium further comprises a cytokinin selected from the group consisting of thidiazuron (TDZ), N-(2-Chloro-4-pyridyl)-N′-phenylurea (4-CPPU), zeatin, and auxin.

23. The method of claim 20, wherein the Eucalyptus explant is an explant selected from the group consisting of: a leaf explant, a petiole explant, an internode explant, a floral tissue explant, or an embryogenic tissue explant.

24. The method of claim 20, wherein the Eucalyptus explant is an explant that has been contacted with an Agrobacterium strain harboring a vector capable of transferring a gene to a plant cell.

25. The method of claim 20, wherein the Eucalyptus explant is a transformed explant.
